(19) United States
(12) Patent Application Publication
(10) Pub. No.: US 2026/0338751 A1
(43) Pub. Date: Oct. 29, 2026

(54) A METHOD OF CALIBRATION FOR CONTINUOUS MONITORING OF METHANE GAS FUGITIVE EMISSIONS

(71) Applicant: SCHLUMBERGER TECHNOLOGY CORPORATION, Sugar Land, TX (US)

(72) Inventors: Albert Ballard ANDREWS, Cambridge, MA (US); Andrew J. SPECK, Cambridge, MA (US); Mathieu DAUPHIN, Cambridge, MA (US); Aditi CHAKRABARTI, Cambridge, MA (US)

(21) Appl. No.: 19/110,842

(22) PCT Filed: Sep. 12, 2023
(86) PCT No.: PCT/US2023/032455

§ 371 Date: Mar. 12, 2025

Related U.S. Application Data
(60) Provisional application No. 63/375,575, filed on Sep. 14, 2022.

(51) Int. Cl. G01N 33/00; G01N 27/12; G06N 3/0464; G06N 3/048

(52) U.S. Cl. CPC G01N 33/0047; G01N 27/12; G01N 33/0006; G06N 3/0464; G06N 3/048

(57) ABSTRACT

Embodiments presented provide for a method of monitoring emissions. A calibration of a metal oxide sensor is accomplished in order to monitor fugitive methane gas emissions on a consistent and constant basis.

### CROSS-REFERENCE TO RELATED APPLICATIONS

The present application claims priority to U.S. Provisional Patent Application 63/375,575 filed Sep. 14, 2022, the entirety of which is incorporated by reference.

### FIELD OF THE DISCLOSURE

Aspects of the disclosure relate to monitoring of emissions. More specifically, aspects of the disclosure relate to a method of calibration of sensors for continuous monitoring of fugitive gas emissions.

### BACKGROUND

Identification and quantification of environmental contaminants in the environment is becoming more important as companies and nations seek to cut air pollution. Historically, methane leaks were allowed in oil field service operations as remediation of these leaks were generally economically prohibitive. These practices; however, have come under regulatory scrutiny as the environmental damage that is potentially caused may be far more ranging than originally thought. National, State and local authorities have implemented or are implementing restrictions on air pollution from various sources.

With the advent of regulatory attempts to curb greenhouse gas emissions, methane gas, specifically, has come under increasingly stringent review. Current methods for identification of methane leaks are based upon conventional fluid dynamics equations. Unfortunately, placements of sensors, variability of environmental conditions, and other constraints hinder the overall ability of operators to identify and quantify methane leaks in the field to levels currently desired.

Conventional sensors that are used in the field to identify methane emissions are based upon known technologies. Such technologies include, but are not limited to linear regression technologies. Such technologies include identification of a point source and the ultimate distance of where monitoring is to be performed. As the gasses travel from the point source, they are diluted by the environment. As can be understood, the further away a sampling point for contamination is from the origination point, the weaker the measured levels. While the linear regression technologies are well known, linear regression is often not the best alternative for identification of contaminants. These technologies may give false readings that are not accurate to precisions needed. For example, many regulatory rules on emitters require that emissions off an owners property must be below a specific threshold. Thus, the important point for monitoring is not at the origination point of the contamination, but rather at the property boundary. It is therefore imperative to understand the flow patterns and travel of contaminants rather than assuming a linear regression dispersion of effluents.

Globally and within the United States, oil and gas operators are currently under scrutiny by government authorities such as the US Environmental Protection Agency (EPA) to reduce their annual contributions of greenhouse gas (GHG) emissions. Methane is the main component of natural gas and a much more potent GHG than carbon dioxide. Equipment installed in the field eventually degrades, however, and leak paths are sometimes inevitable, even with the most proven technologies. Detecting methane emissions is useful for measuring and monitoring leaks. Operators are required to perform inspections at varying frequencies throughout the year which can be expensive. Moreover, these inspections do not provide the client with an accurate measurement of leaks in an integrated fashion both in space and time.

Ideally, continuous monitoring of methane emissions by sensors installed permanently in the operator locations could overcome the above-mentioned limitations. To accomplish the goal of continuous monitoring followed by mitigation; methane monitors should be sensitive, quantitative, fast, accurate and inexpensive. None of the existing methane sensors in the market; however, qualify as the perfect candidate for field deployment in continuous methane monitoring activities.

Metal oxide semiconductor (MOx) sensors detect adsorbed target gases which reduce atmospheric oxygen on the SnO₂ surface, lowering the resistance when electrons are added to the conduction band. MOx sensors are sensitive to relative humidity (RH) when water is absorbed as well as temperature (T) which changes the reaction rates. As a consequence, calibrations of the sensors must be conducted in an environmental chamber where both parameters are accurately controlled. The calibration data is collected from the MOx sensor in parallel with a benchtop sub-ppm optical methane analyzer, ramping the methane concentration at each step from 2-2500 ppm at different relative humidity (RH) and temperature (T) settings (see, FIG. 4). The dynamic range can be extended beyond specifications by utilizing multiple frequencies in a resonant AC circuit (see, FIG. 2).

There is a need to provide an apparatus and methods that accurately measure methane emissions for owners of industries that may potentially emit fugitive gas emissions.

There is a further need to provide apparatus and methods that do not have the drawbacks discussed above, namely the inherent limitations of linear regression techniques.

There is a still further need to reduce economic costs associated with operations and apparatus for identification of methane contaminants described above with conventional tools.

### SUMMARY

So that the manner in which the above recited features of the present disclosure can be understood in detail, a more particular description of the disclosure, briefly summarized below, may be had by reference to embodiments, some of which are illustrated in the drawings. It is to be noted that the drawings illustrate only typical embodiments of this disclosure and are therefore not to be considered limiting of its scope, for the disclosure may admit to other equally effective embodiments without specific recitation. Accordingly, the following summary provides just a few aspects of the description and should not be used to limit the described embodiments to a single concept.

In one example embodiment, a method for calibrating a sensor for continuous monitoring of methane gas emissions is disclosed. The method comprises acquiring calibration data for a reference sensor placed within an environmental chamber. The method further comprises acquiring an impedance for the sensor under known conditions. The method further comprises training a regression model with training data to produce model calibration results. The method further comprises comparing the acquired calibration data to the model calibration results. The method further comprises when the comparing the acquired calibration data is within a threshold value to the model calibration results, transferring the model calibration results to at least one sensor.

In another example embodiment, a method for calibrating a sensor for continuous monitoring of methane gas emissions is disclosed. The method may comprise acquiring calibration data for a reference sensor. The method may further comprise calculating real and imaginary impedances for the reference sensor based upon the acquired calibration data. The method may further comprise training a neural network with at least two predictor values to produce calibration model results. The method may further comprise comparing the calibration data for the reference sensor to the calibration model results. The method may further comprise when the comparing of the calibration data for the reference sensor to the calibration model results is within a threshold value, transferring the calibration model results to at least one sensor.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the drawings. It is to be noted; however, that the appended drawings illustrate only typical embodiments of this disclosure and are therefore not be considered limiting of its scope, for the disclosure may admit to other equally effective embodiments.

FIG. 1 is a workflow of procedural steps for calibration of sensors in one example embodiment of the disclosure.

FIG. 2 is a resonant resistance circuit used for modeling MOx sensors.

FIG. 3 are graphs of impedance versus frequency for real and imaginary parts of different methane concentrations.

FIG. 4 is a calibration run for a sensor illustrating steps in temperature and relative humidity or absolute humidity and ramps of methane concentration.

FIG. 5 is a graph of resistance in ohms versus ppm concentration of methane.

FIG. 6 is a graph of measured and predicted methane concentration for a range of 2 to 2500 parts per million (ppm) for one example embodiment of the disclosure.

FIG. 7 is a graph of a shallow neural network with 3 inputs in one example embodiment of the disclosure.

FIG. 8 is a plot of measured and predicted methane for training and test set s of data.

FIG. 9 is a flowchart illustrating an architecture for a deep learning network in one example embodiment of the disclosure.

FIG. 10 is a plot of measured versus predicted ppm methane using a gradient boosted tree ensemble.

FIG. 11 is an overlay of response of an optical analyzer with a MOx sensor from a field test.

FIG. 12 is a correlation plot of response of an optical analyzer vs a MOx sensor from a field test.

To facilitate understanding, identical reference numerals have been used, where possible, to designate identical elements that are common to the figures (“FIGS”). It is contemplated that elements disclosed in one embodiment may be beneficially utilized on other embodiments without specific recitation.

### DETAILED DESCRIPTION

In the following, reference is made to embodiments of the disclosure. It should be understood; however, that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments and advantages are merely illustrative and are not considered elements or limitations of the claims except where explicitly recited in a claim. Likewise, reference to “the disclosure” shall not be construed as a generalization of inventive subject matter disclosed herein and should not be considered to be an element or limitation of the claims except where explicitly recited in a claim.

Although the terms first, second, third, etc., may be used herein to describe various elements, components, regions, layers and/or sections, these elements, components, regions, layers and/or sections should not be limited by these terms. These terms may be only used to distinguish one element, components, region, layer or section from another region, layer or section. Terms such as “first”, “second” and other numerical terms, when used herein, do not imply a sequence or order unless clearly indicated by the context. Thus, a first element, component, region, layer or section discussed herein could be termed a second element, component, region, layer or section without departing from the teachings of the example embodiments.

When an element or layer is referred to as being “on,” “engaged to,” “connected to,” or “coupled to” another element or layer, it may be directly on, engaged, connected, coupled to the other element or layer, or interleaving elements or layers may be present. In contrast, when an element is referred to as being “directly on,” “directly engaged to,” “directly connected to,” or “directly coupled to” another element or layer, there may be no interleaving elements or layers present. Other words used to describe the relationship between elements should be interpreted in a like fashion. As used herein, the term “and/or” includes any and all combinations of one or more of the associated listed terms.

Some embodiments will now be described with reference to the figures. Like elements in the various figures will be referenced with like numbers for consistency. In the following description, numerous details are set forth to provide an understanding of various embodiments and/or features. It will be understood; however, by those skilled in the art, that some embodiments may be practiced without many of these details, and that numerous variations or modifications from the described embodiments are possible. As used herein, the terms “above” and “below”, “up” and “down”, “upper” and “lower”, “upwardly” and “downwardly”, and other like terms indicating relative positions above or below a given point are used in this description to more clearly describe certain embodiments.

Aspects of the disclosure describe a method to deploy an MOx sensor for detecting trace concentrations of methane, with high accuracy, which is cost-effective, and differentiated from existing, ready-to-use, MOx sensor technologies. To report accurate values of methane emissions, one step in the disclosure is calibrating these MOx sensors. Upon the calibration, the sensor readings can be converted to respective concentration values. The following content describes methods to calibrate the MOx gas sensors used for continuous monitoring activities.

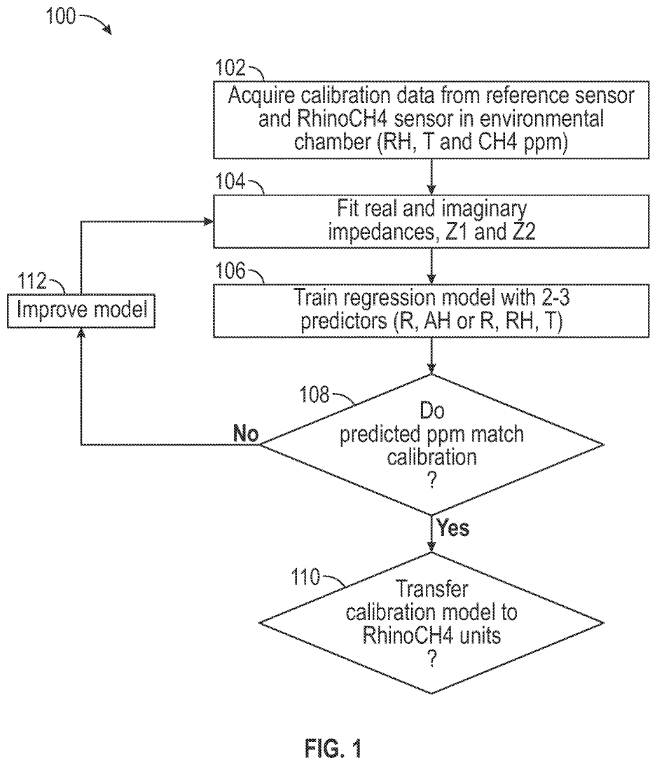

Referring to FIG. 1, a method 100 of performing a calibration for continuous monitoring of methane gas fugitive emissions is illustrated. At 102, the method involves acquiring calibration data from a reference sensor in an environmental chamber. At 104, the method continues where real and imaginary impedances are fit to the corresponding calibration data. At 106, the method further provides for training a regression model with two or three predictors. Such predictors can be, in non-limiting embodiments, for two predictors, R and AH, and for three predictors, R, T and RH. Note: See page 3 of Template for Instructions. At 108, the method also provides for a test, where it is determined if the predicted calibration matches the actual calibration. At 110, if there is a match between the predicted calibration and the actual calibration, then the method also provides for transferring the calibration model to field sensors. If there is no match, then the method calls for, at 112, improving the model at 112, and rerunning the model at 102. The number of times that the method 100 may be run, may be according to the establishment of a threshold value. For example, the method 100 may be continually run until the predicted values and actual calibration values are below 0.0001 percent. Other values may be used for the threshold value and, as such, a user may define the ultimate resolution necessary.

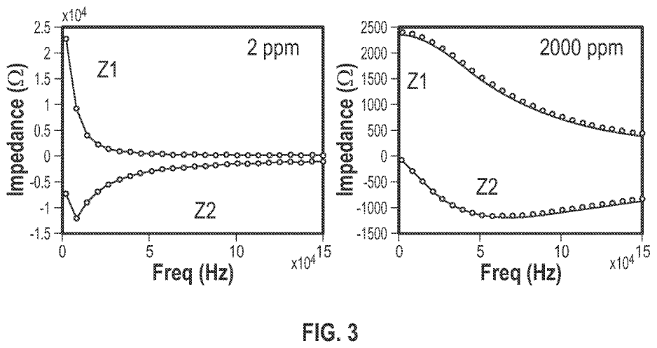

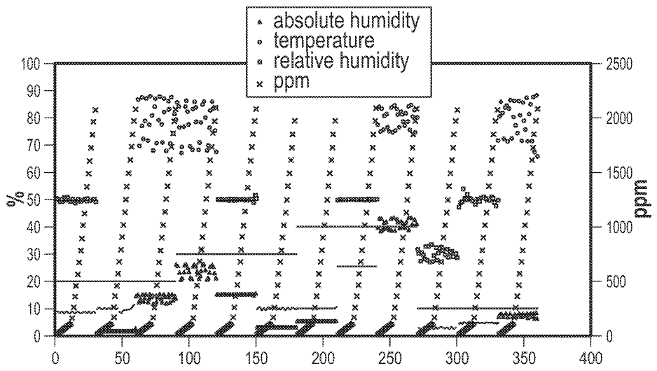

In accordance with the method described above in relation to FIG. 1, Equation 1 (below), shows the formulas for the real (Z1) and imaginary (Z2) parts of the impedance for a parallel RC circuit such as that used to represent a MOx sensor. The measured impedance curves versus frequency, as shown in FIG. 3, are fit using a non-linear, least squares, algorithm. The result is a set of R (resistivity) curves for each absolute humidity (AH) and/or RH, T step as a function of parts per million concentration of methane, as illustrated in FIG. 4.

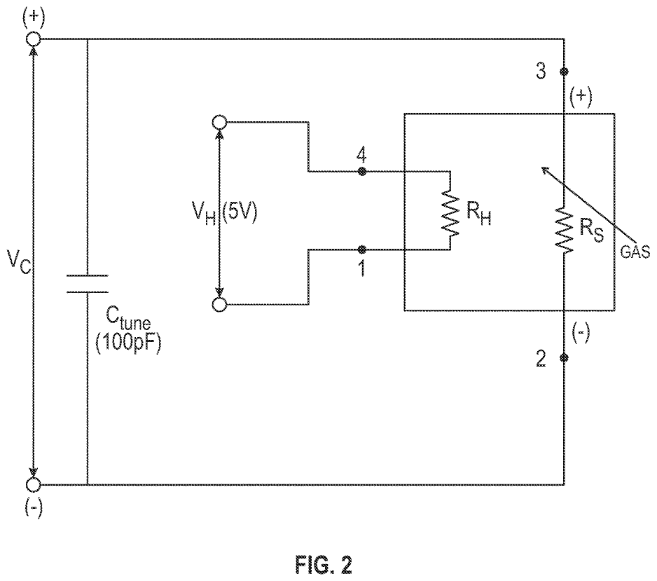

In embodiments, a Gaussian Process Regression Model (GPR) is used. The predictor inputs for the Gaussian Process Regression Model are drawn from the fitted R or measured R and either the AH or R and RH, T curves. Note that the values of temperature (T) and relative humidity (RH) may be transformed algebraically (see, for example, Equation 2) to absolute humidity (AH). The predicted concentration of methane in parts per million (ppm) is similar, regardless of whether the values of AH or RH and T are used as predictors. Referring to FIG. 2, a sample resonant resistance circuit for and MOx is illustrated. This sample resonant resistant circuit may be used in conjunction with MOx sensors for calibration purposes. To perform this evaluation, an impedance analyzer is connected across the VC (+) and VC (−) points indicated. Referring to FIG. 3, a plot is illustrated that fits the real and imaginary parts, calculated using Equation 1, below, of the impedance at different methane concentrations.

$$Real {Z}_{1} = \frac{R}{1 + {( 2 π fCR )}^{2}} Imaginary {Z}_{2} = \frac{- {R}^{2} C 2 π f}{1 + {( 2 π fCR )}^{2}} \qquad Equation 1$$

AH=(6.112*exp((17.67*T)/(T+243.5))*RH*2.1674)/(273.15+T)) Equation 2

Referring to FIG. 4, a typical calibration run, in accordance with one example embodiment of the disclosure, is illustrated. In this embodiment, data that is shown that includes temperature (degrees c) and relative humidity percent (darkest scattered circles), or equivalent absolute humidity percent (lightest scattered circles) as well as ramps in methane concentration ppm (x), at each step. The steps for calibration at 2 ppm and 2500 ppm are illustrated. As will be understood, calibration may be conducted at other levels in instances where different concentrations are needed for identification.

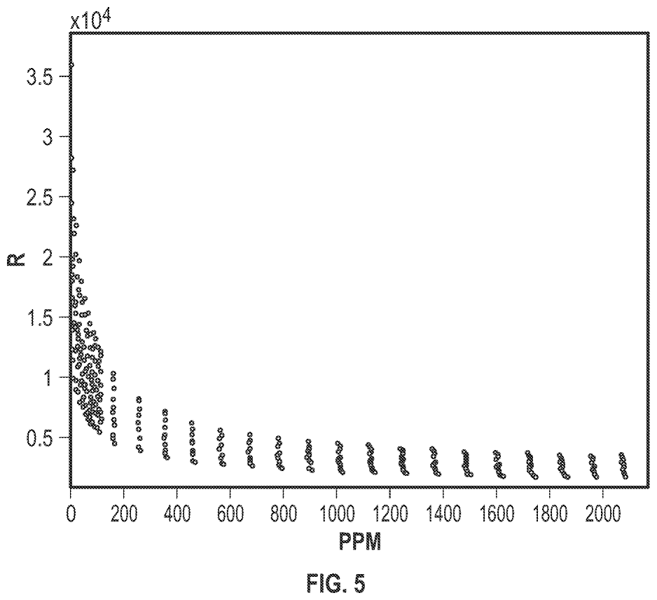

Referring to FIG. 5, the resistance R (ohm) is obtained by fitting Equations 1 and 2 for the real and imaginary impedance for all ppm, AH and/or T and RH. FIG. 5 shows resistance R as a function of methane concentration.

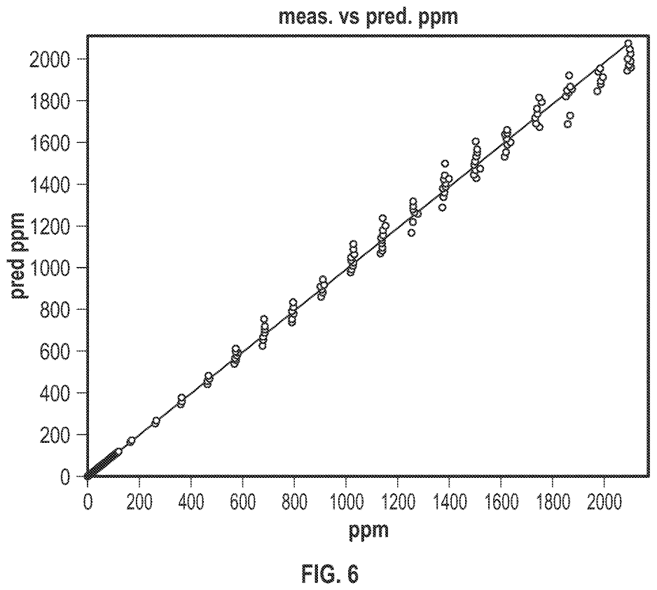

Referring to FIG. 6, a non-limiting example plot of measured methane concentration (X) versus predicted methane concentration (Y) is illustrated. As can be seen, the relationship is essentially a straight line, as expected. Scatter values are less for smaller concentrations of methane as opposed to larger concentration values, therefore the Mean Average Error (MAE) between the measured and predicted ppm decreases

### Machine Learning

Chemometrics methods such as partial least squares are appropriate for many spectroscopic applications may be used to establish machine learning algorithms for help in calibration methods in non-limiting embodiments of the disclosure. Machine learning methods include, but are not limited to, neural networks, support vector machines, regression trees, ensembles of trees, and Gaussian Process Regression models. The disclosed methods should not be considered limiting. For use in the models described above, the predictor inputs are R, AH or R, T, RH, and the response is the CH₄ concentration in parts per million (ppm) of methane.

### Neural Networks

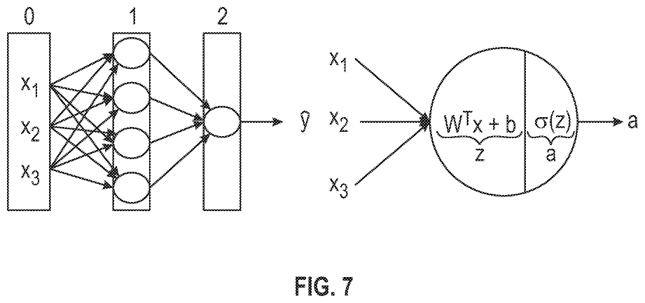

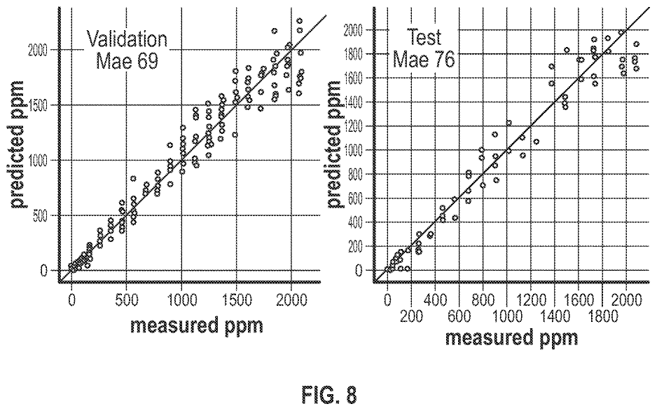

Shallow neural networks with a single or multiple hidden layers and a moderate number of nodes (10-100) are computationally less demanding than deep learning networks. As such, shallow neural networks may be used to efficiently calculate concentration values needed. Referring to FIG. 3, the neural network in FIG. 7 (using Equation 3) has three input features a⁰ⱼ=xⱼ, four nodes in a hidden layer, and an output defined as a²=ŷ. The difference between ŷ and the true value of y is minimized with respect to the parameters wⱼₖ (weights) and bⱼ (bias) using a regression analysis. The response function, defined as g^{|I|}, may be the sigmoid s(z)=(1+e^{-z})⁻¹ or tanh or a rectified linear unit activation (ReLU) function. Other values may be used. FIG. 8 graphs the true concentration in ppm versus predicted concentration in ppm for the validation and test data sets using holdout cross validation methods. In one non-limiting embodiment, the mean average errors (MAE) values are achieved for a bi-layer neural network with ten nodes in two hidden layers using a ReLU function.

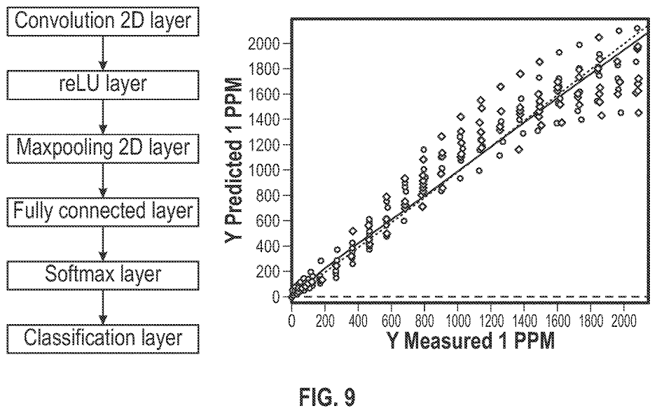

Other neural networks may also be used. In one non-limiting embodiment, a deep learning network is used. Examples of such deep learning networks include, such as TensorFlow with an optimizer Adam (Adaptive Moment Estimation), AdaMax (an extension of Adaptive Movement Estimation), RMSProp (Root Mean Squared Propagation), SGD (Stochastic Gradient Descent) and a layer type, deep, learning, network (having alternatives of dense, dropout, flattened, 1D, 2D, or 3D convolutional, and/or pooling layers). Alternatively, a program entitled Scikit-Learn may be used with an appropriate solver (Adam, Ibfgs or SGD) and activation (ReLU, identity, tanh or logistic). In this aspect of the disclosure used herein for results, the framework is TensorFlow with an Adaptive Moment Estimation optimizer (Adam). In the embodiments shows, running averages are computed for both the gradients and the second moments of the gradients. In these embodiments an activation layer Rectified Linear Unit, and dense or fully connected layer is used, whose neurons connect to every neuron in the preceding layer as shown in FIG. 9.

Referring to FIG. 7, a shallow neural network used in one example embodiment for calculation of values for concentration has three (3) input features in layer 0, four nodes in the hidden layer 1, and one node in the output layer 2.

$${z}_{j}^{[ ℓ ]} = {\sum }_{k = 1}^{{n}^{[ ℓ ]}} {w}_{jk}^{[ ℓ ]} {a}_{k}^{[ ℓ - 1 ]} + {b}_{j}^{[ ℓ ]} ; {a}^{[ ℓ ]} = {g}^{[ ℓ ]} ( {z}_{j}^{[ ℓ ]} ) \qquad Equation 3$$

Referring to FIG. 8, an example plot of measured concentrations of methane in ppm (X) versus predicted measured concentrations of methane in ppm (Y) is shown. To ensure accuracy, training sets of data are used (ths) and compared to a test set of data (rhs) as predicted by the neural network. In this embodiment, ReLU activation was used.

Referring to FIG. 9 (left side), a flowchart is provided that illustrates an example architecture for a deep learning network for layers used for calculations. Such an example architecture should not be considered limiting. The ordering of layers used for analysis may include, a convolution 2D layer, a ReLU layer, a maxpooling 2D layer, a fully connected layer, a softmax layer and a classification layer. The graph in the right side of FIG. 9, shows a plot of predicted concentrations versus measured concentrations of methane concentration using a deep learning network. The training set data is noted by circular points. Test set data is noted by diamond points. The type of deep learning network used is a TensorFlow network with the Adam optimizer and a ReLU activation layer. For this example, a fully connected layer with 100 nodes was used.

### Ensemble Methods of Machine Learning

In some embodiments, machine learning may be accomplished through the use of ensemble methods of machine learning. In such ensemble methods of machine learning, multiple types of learning algorithms are used, as compared with some embodiments previously disclosed with a single algorithm. Through testing, it has been found that using multiple types of learning algorithms provides a superior result compared to a single learning algorithm. One type of ensemble method known is a gradient boosted tree ensemble. By using a gradient boosted tree ensemble, acceptable accuracies are achieved, especially at lower concentrations (<500 ppm).

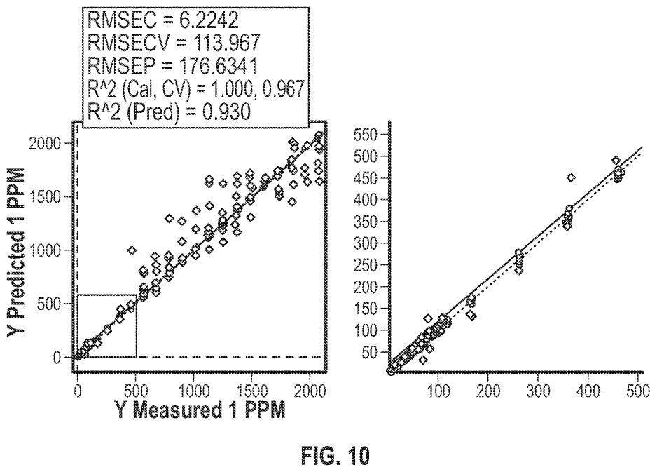

Referring to FIG. 10, a plot of measured concentrations of methane in ppm (X) versus predicted concentrations of methane in ppm (Y) is illustrated using a gradient boosted tree ensemble, described above. As in the previous example, the training set is noted through circle data point representation and the test set of data is noted by diamond point representation. In this embodiment, a Kennard-Stone algorithm (66/34) is used with RMSEP=171, R^{\hat{ }2}=0.93 (rhs); range 0-500 ppm (lhs).

### Gaussian Process Regression

In further embodiments of the disclosure, a gaussian progress regression may be used for calibration purposes. A Gaussian Process (GP) is a regression and classification technique that infers how data is correlated, predicting a distribution and associated uncertainty. A Gaussian Process is a generalization of a Gaussian probability distribution to infinitely many variables, defined by a mean m(x) and covariance function k(x, x′), or f(x)~GP(m(x), k(x, x′)). The covariance function (kernel) between two or more latent variables e.g., R, and T, RH, or AH, determines how the response at a point is affected by the response at other points.

In one example embodiment of a Gaussian Process regression is disclosed in Equation 4, recited below. Equation 4 is a covariance function, a double exponential, also known as, a radial basis function. Other functions for Gaussian Process Regression may be used, such as functions with a single predictor. In embodiments, automatic relevance determination (ARD) may be used to separate length scales for each predictor. When automatic relevance determination is used, Equation 5 may be used.

$$k ( {x}_{i} , {x}_{j} \vert θ ) = {σ}_{f}^{2} exp [ - \frac{1}{2} \frac{{( {x}_{i} - {x}_{j} )}^{T} ( {x}_{i} - {x}_{j} )}{{σ}_{l}^{2}} ] . \qquad Equation 4$$

$$k ( {x}_{i} , {x}_{j} \vert θ ) = {σ}_{f}^{2} exp [ - \frac{1}{2} {\sum }_{m = 1}^{d} \frac{{( {x}_{im} - {x}_{jm} )}^{2}}{{σ}_{m}^{2}} ] . \qquad Equation 5$$

The kernel parameters of Equation 4 listed above are the signal standard deviation σ_{f} and a characteristic length scale σ₁. In instances where the inputs are close to each other, the covariance is close to one and decreases exponentially as the distance increases. The hyperparameters in the covariance kernel may be tuned in some embodiments.

Once the training is completed, the Gaussian Progress regression model is cast into a structural array which is applied to the data streams from MOx sensors deployed around the well pads. The real and imaginary impedances (and frequencies) are passed through a series of function calls, which return the predicted concentration of methane in ppm for each data point.

As an example of a Gaussian Progress regression model, 1.) the resistance is fit using a least squares method or is measured. After the least squares method, a second step of applying a scaling factor (mean center) and offset (standard deviation) is accomplished, defined as “autoscaling”. This autoscaling is applied to the current data points Ri, Ti and RHi triplet as seen in Equation 6 recited below:

RTRHi′=gpr.xscale.\([Ri,Ti,RHi]−gpr.xoffset) Equation 6

In a third step, an (exponential) kernel predicts the methane concentration value in ppm for the current data point according to Equation 7, recited below:

covariance kernel=gpr.σ_{f}\hat{ }2*exp(−d(xi,xj)/gpr.σ₁) Equation 7

Where the values of gpr.σ_{f} is the signal variance, gpr.σ₁ is the length scale. A distance d may be defined per Equation 8, as is the Euclidean distance between the new data points and the points in the calibration model:

d(xi,\sqrt{(xᵢ−xⱼ)^{T}(xᵢ−xⱼ)}xj)= Equation 8

The predicted concentration of method in ppm for the current data point is given by Equation 9:

ppm=covariance kernel(RTRHi′,grp.x)′*gpr.α Equation 9

where the value grp.x is the data in the calibration model and gpr.α is the variance of Gaussian measurement noise on the training observations.

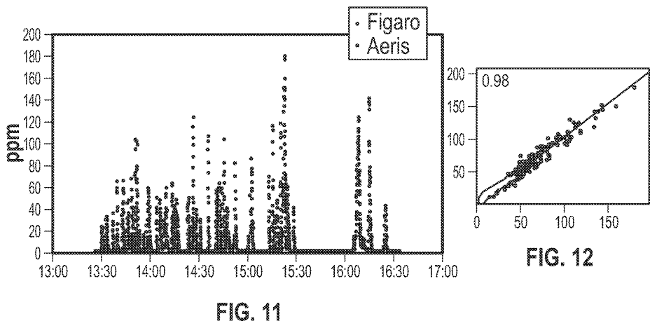

FIGS. 11 and 12 compare the response from a commercial optical analyzer and a calibrated MOx sensor during a controlled leak test. A correlation of 0.98 between the two signals is a compelling demonstration of the value of using a machine learning approach for MOx sensor calibration.

The mathematical calculations, as shown above, may be performed on a computer. Embodiments of the disclosure provide for incorporating the method steps, and alterations of the method steps in FIG. 1, onto a non-volatile medium that may be configured to run on a computer. Such non-volatile medium may be, for example, a compact disk, a universal serial bus device, a solid-state memory or other similar devices. Calculations may also be performed through computers connected through web devices or cloud devices.

For purposes of illustration, example embodiments, described in the claims, are recited herein. Such recitation of the embodiments should not be considered limiting. In one example embodiment, a method for calibrating a sensor for continuous monitoring of methane gas emissions is disclosed. The method comprises acquiring calibration data for a reference sensor placed within an environmental chamber. The method further comprises acquiring an impedance for the sensor under known conditions. The method further comprises training a regression model with training data to produce model calibration results. The method further comprises comparing the acquired calibration data to the model calibration results. The method further comprises when the comparing the acquired calibration data is within a threshold value to the model calibration results, transferring the model calibration results to at least one sensor.

In another example embodiment, the method may be performed wherein the calculating impedances involves calculating a real and imaginary portion for the impedances.

In another example embodiment, the method may be performed wherein the regression model is a computer model.

In another example embodiment, the method may be performed wherein the computer model involves one of a Gaussian Process Regression or a neural network.

In another example embodiment, the method may be performed wherein the training data includes at least one predictor

In another example embodiment, the method may be performed wherein there are at least two predictors.

In another example embodiment, the method may be performed, wherein there are least three predictors.

In another example embodiment, the method may be performed wherein when the comparing the acquired calibration data is not within the threshold value, improving the model and performing the method again.

In another example embodiment, a method for calibrating a sensor for continuous monitoring of methane gas emissions is disclosed. The method may comprise acquiring calibration data for a reference sensor. The method may further comprise calculating real and imaginary impedances for the reference sensor based upon the acquired calibration data. The method may further comprise training a neural network with at least two predictor values to produce calibration model results. The method may further comprise comparing the calibration data for the reference sensor to the calibration model results. The method may further comprise when the comparing of the calibration data for the reference sensor to the calibration model results is within a threshold value, transferring the calibration model results to at least one sensor.

In another example embodiment, the method may be performed wherein at least one of the at least two predictor values is a temperature, an actual humidity, and a relative humidity.

In another example embodiment, the method may further comprise altering at least one calculation value in the neural network and performing the method again when the comparing of the calibration data is not within the threshold value.

In another example embodiment, the method may further comprise inputting the threshold value through a user interaction.

In another example embodiment, the method may be performed wherein the hyperparameters of the GPR are isotropic, wherein the length scales for each predictor are the same.

In another example embodiment, the method may be performed wherein the hyperparameters of the GPR are non-isotropic, wherein the length scales for each predictor are not the same. In another embodiment, automatic relevance determination (ARD) may be used to separate length scales for each predictor.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may be varied in many ways. Such variations are not to be regarded as a departure from the disclosure, and all such modifications are intended to be included within the scope of the disclosure.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

What is claimed is:

1. A method for calibrating a sensor for continuous monitoring of methane gas emissions, comprising:
acquiring calibration data for a reference sensor placed within an environmental chamber;
acquiring an impedance for the sensor under known conditions;
training a regression model with training data to produce model calibration results;
comparing the acquired calibration data to the model calibration results; and
when the comparing the acquired calibration data is within a threshold value to the model calibration results, transferring the model calibration results to at least one sensor.

2. The method according to claim 1, wherein the calculating impedances involves calculating a real and imaginary portion for the impedances.

3. The method according to claim 1, wherein the regression model is a computer model.

4. The method according to claim 3, wherein the computer model involves one of a machine learning model such as a neural network or a Gaussian Process Regression model.

5. The method according to claim 1, wherein the training data includes at least one predictor.

6. The method according to claim 5, wherein there are more than one predictor.

7. The method according to claim 1, wherein when the comparing the acquired calibration data is not within the threshold value, improving the model and performing the method again.

8. A method for calibrating a sensor for continuous monitoring of methane gas emissions, comprising:
acquiring calibration data for a reference sensor;
calculating real and imaginary impedances for the reference sensor based upon the acquired calibration data;
training a Gaussian Process Regression model with at least two predictor values to produce calibration model results;
comparing the calibration data for the reference sensor to the calibration model results; and
when the comparing of the calibration data for the reference sensor to the calibration model results is within a threshold value, transferring the calibration model results to at least one sensor.

9. The method according to claim 8, wherein at least one of the at least two predictor values is a temperature, an actual humidity, and a relative humidity.

10. The method according to claim 8, further comprising:
altering at least one calculation value in the Gaussian Process Regression; and
performing the method again when the comparing of the calibration data is not within the threshold value.

11. The method according to claim 9, further comprising inputting the threshold value through a user interaction.

12. The method according to claim 9, wherein the Gaussian Process Regression is isotropic

13. The method according to claim 9, wherein the Gaussian Process Regression model is non-isotropic.
